(19) United States
(12) Patent Application Publication
(10) Pub. No.: US 2021/0369884 A1
(43) Pub. Date: Dec. 2, 2021

(54) UV-C STERILIZER ATTACHABLE TO INPUT PAD OF AUTOMATED MACHINE

(71) Applicant: HADELSYS CO., LTD., Siheung-si (KR)

(72) Inventor: Won Gee LEE, Seoul (KR)

(21) Appl. No.: 16/891,553

(22) Filed: Jun. 3, 2020

(30) Foreign Application Priority Data
May 26, 2020 (KR) 10-2020-0062735

(51) Int. Cl. A61L 2/10; A61L 2/24; A61L 2/26

(52) U.S. Cl. CPC A61L 2/10; A61L 2/24; A61L 2/26; G07F 19/201; A61L 2202/14; A61L 2202/16; A61L 2202/11

(57) ABSTRACT

Provided is a sterilizer simply attached to the edges of an input pad of an existing automated machine, wherein a moving UV bar (20) having a UV-C light source is vertically moved by a driving means to sterilize the surface of the input pad. The sterilizer includes: a base plate (10) having an open portion (11) and attachable to edges of the input pad (2); the moving UV bar (20) having the light source (21) in which multiple high-power UV-C LEDs are arranged at predetermined intervals on a longitudinal bar, and a protection cover (22); a horizontal cover (14) provided in a horizontal direction above the open portion and vertical covers (13L and 13R) provided at opposite sides of the open portion (11); and the driving means configured to be compactly arranged inside a space defined by the covers (13L, 13R, and 14) and the base plate (10).

### BACKGROUND OF THE INVENTION

### Field of the Invention

The present invention relates generally to a sterilizer attachable to an input pad of an ATM, a kiosk, or a vending machine, etc. More particularly, the present invention relates to a sterilizer attachable to an input pad of an automated machine (an ATM, a kiosk, a vending machine, etc.) which is currently used in many places for various purposes without being embedded in a newly manufactured automated machine (an ATM, a kiosk, or a vending machine, etc.).

### Description of the Related Art

Recently, a vending machine, through which a user can directly order or purchase goods without the help of a salesperson, is used in various fields. There are many types of vending machines, such as ATMs for financial transactions, kiosks through which consumers receive services or goods which they want to buy after paying for the services or goods at theaters or markets, and vending machines, through which you can buy drinks or cigarettes. Hereinafter, such machines are referred to as “automated machines”.

All such automated machines have an input pad, through which a desired service or item is selected and various information required for ordering is input. The input pad may be a button or a touch screen. For example, an input pad in an ATM is also called an encrypting pin pad (EPP).

Automated machines are used by a large number of people, and it is very likely that bacteria or viruses get on the input pad of each of the automated machines touched by many users, so these bacteria can reproduce on the input pad or spread to the next users.

In particular, there is a high risk that many influenza viruses or bacteria, including the recently spreading coronavirus (COVID-19), will infect numerous users through the input pad of the automated machine. “Automated machines” mentioned in the present invention include an elevator in which many input buttons are provided.

A number of conventional arts for removing viruses and bacteria by emitting a predetermined amount of ultraviolet light on the surface of the input pad of a keyboard, a smartphone, an ATM, or a kiosk have been disclosed.

Although the ability of UV-C's to remove viruses is well known, UV-C should not be exposed to human skin or eyes because it is known to cause carcinogenesis and cataracts in mammals. However, it is known that UV-C having a specific wavelength (207 to 222 nm) among UV-C can efficiently remove viruses without harming mammalian skin. However, it is clear that LEDs emitting UV-C having this specific wavelength (207 to 222 nm) have not been developed yet, and will be very expensive even if developed in the near future.

Therefore, it is necessary to use a UV-C LED emitting the light of a wavelength of 265 to 280 nm, which is known to have the best sterilization effect, but the UV-C LED is required to be developed in a structure in which UV-C is prevented from being emitted on the human body (particularly, the eyes and hands).

In the process in which a sterilizer attached to the automated machine normally operates, UV-C light is required to be prevented from being emitted on a portion (for example, the fingers, the face, or the eyes, etc.) of the body of a user, and to be emitted to be focused only on a target (the surface of the input pad) to be sterilized so that the operation time of the sterilizer is minimized and sterilization efficiency is maximized. In addition, measures to protect a user who does an abnormal action (for example, the action of pushing the fingers between the UV-C light source and the input pad) from UV-C should also be considered.

Furthermore, from a point of view, it is preferable that a UV-C sterilizer is configured to be embedded inside the casing of the automated machine (an ATM, a kiosk, a vending machine, etc.). However, since the automated machine (an ATM, a kiosk, a vending machine, etc.) is used in many places for various purposes, a sterilizer having a simple structure capable of performing sterilization by being simply attached to the input pad of the existing automated machine without replacing the automated machine is required.

### DOCUMENTS OF RELATED ART

### (Patent Document 1) Korean Utility Model Application Publication No. 20-2011-0002728;

### (Patent Document 2) Korean Patent No. 10-1158046;

### (Patent Document 3) Korean Patent Application Publication No. 10-2011-0089385;

### (Patent Document 4) Korean Patent No. 10-1132762;

(Patent Document 5) U.S. Pat. No. 10,596,281; and

### (Patent Document 6) U.S. Patent Application Publication No. 2015/0174276

### NON-PATENT DOCUMENTS

(Non-patent Document 1) American Society Microbiology “UVC LED irradiation effectively inactivates aerosolized viruses, bacteria, and fungi in a chamber-type air disinfection system” (Published 29 Jun. 2018), and
(Non-patent Document 2) Nature, Scientific Reports “Far-UVC light: A new tool to control the spread of airborne-mediated microbial diseases” (Published 09 Feb. 2018)

### SUMMARY OF THE INVENTION

Accordingly, the present invention has been made keeping in mind the above problems occurring in the related art, and the present invention is intended to propose a UV-C sterilizer which is simply attached to the edges of an input pad of an automated machine.

The present invention is further intended to propose a UV-C sterilizer that can protect a user from UV-C light while sterilizing an input pad of an automated machine by irradiating the surface of the input pad of the automated machine with UV-C light of a wavelength range harmful to the human body

The present invention is still further intended to propose a sterilizer that in the case that many people are waiting to use an automated machine, allows a sterilization operation to be completed during a short time when a next user approaches the automated machine after a previous user finishes using the automated machine so that the next user does not wait during time required to sterilize the input pad, thereby minimizing transaction time for sterilizing and maximizing sterilization efficiency.

The present invention is still further intended to provide a sterilizer having high durability that is not easily broken by abnormal manipulation or external forces.

In order to achieve the above objectives, according to one aspect of the present invention, there is provided a sterilizer including: a base plate having an open portion corresponding to an input pad of an automated machine and attachable to edges of the input pad; a moving UV bar having a light source emitting UV-C toward the open portion of the base plate and vertically moving; and a driving means provided at each of opposite ends of the moving UV bar, the driving means vertically moving the moving UV bar such that the surface of each button of the input pad located at the open portion is sterilized.

The sterilizer according to the embodiment of the present invention may further include a horizontal cover provided in a horizontal direction above the open portion and a vertical cover provided at each of opposite sides of the open portion to protect the driving means.

The moving UV bar of the sterilizer according to the embodiment may include the light source in which multiple high-power UV-C LEDs are integrated to the longitudinal bar, and a protection cover preventing light of the light source from being emitted in directions other than a direction toward a surface of an input pad.

In the sterilizer according to the embodiment of the present invention, a gap defined between a lower edge of the protection cover and the surface of the input pad may be configured to have a size to prevent a finger of a user from entering the gap.

To protect a user, the sterilizer according to the embodiment of the present invention may further include a controller receiving information on whether or not a user is in front of the automated machine from the automated machine, and controlling such that the sterilizer is operated when there is no user.

The controller may be configured to control the sterilizer such that an electric current is applied to the UV-C LEDs only when each part of a driving mechanism, a control board, and a software is recognized to be in a normal state.

The controller may be configured to control the sterilizer such that a status result value of the sterilizer is transmitted to the automated machine after checking whether each part thereof is in a normal state such that information on whether the sterilizer is operated or the maintenance of the sterilizer can be maintained inside the operating system of the entirety of the automated machine.

The sterilizer according to the embodiment of the present invention includes the light source in which the multiple high-power UV-C LEDs are arranged at predetermined intervals on a longitudinal bar and integrated thereto, and allows a gap defined between the light source and the surface of a sterilization target to be optimized, so that a rapid moving speed of the light source is secured and a high sterilization effect is maintained.

In the sterilizer according to the exemplary embodiment of the present invention, to make the driving means compact, a plurality of pinions rotated in the same directions by a drive motor may be provided, and while being moved by rotation of a pinion, a rack may be configured to continue to be rectilinearly moved by a pinion adjacent thereto.

The driving means of the sterilizer according to the embodiment of the present invention may further include a torque limiter preventing damage of components of the automated machine when the movement of the moving UV bar is restricted by impact of a user while the moving UV bar performs UV-C sterilization during the vertical movement. In the sterilizer having the torque limiter, the torque limiter may control so that the moving UV bar is returned to an original position thereof or is moved to a temporary position after a user determines that an operational malfunction occurs when the moving UV bar does not reach a sensor at a specific position by a predetermined time.

Other problems to be solved by the sterilizer according to the present invention and specific means for solving the problems will be described in more detail in the detailed description of the present invention to be described hereinafter.

The UV-C sterilizer according to the present invention is a compact sterilizer, wherein the driving means is compactly provided inside the vertical covers 13L and 13R of the opposite sides of the open portion in the center of the base plate relative thereto and the horizontal cover 14 above the open portion. The sterilizer can be used simply by being attached to the edges of the input pad of an automated machine, which is currently and widely installed and operated, thereby having a high industrial availability.

In addition, the sterilizer according to the present invention contains various user protection measures that can protect a user from UV-C light during sterilization of the input pad of the automated machine, thereby securing economic feasibility and safety.

In the present invention, the light source 21 in which the multiple high-power UV-C LEDs are arranged on the longitudinal bar and integrated thereto is utilized, and a gap defined between the light source and the input pad of the automated machine is optimized. Accordingly, the sterilizer of the present invention allows a sterilization operation to be completed during a short time when a next user approaches the automated machine after a previous user finishes using the automated machine so that the next user does not wait during time required to sterilize the input pad, thereby minimizing transaction time for sterilizing and maximizing sterilization efficiency.

Other effects of the sterilizer according to the present invention will be described in more detail in the detailed description of the invention described below.

### BRIEF DESCRIPTION OF THE DRAWINGS

The above and other objects, features, and other advantages of the present invention will be more clearly understood from the following detailed description when taken in conjunction with the accompanying drawings, in which:

FIG. 1 is a perspective view illustrating a sterilizer of the present invention attached to an input pad of an automated machine;

FIG. 2 is an enlarged perspective view illustrating the sterilizer of the present invention of FIG. 1;

FIG. 3 is an exploded perspective view illustrating the structure of the sterilizer of the present invention;

FIG. 4 is a perspective view illustrating a light source 21 in which the multiple high-power UV-C LEDs of the present invention are arranged on a longitudinal bar and integrated thereto;

FIG. 5 is a view illustrating the interrelationship of a distance between the surface of a sterilization target and each LED, and an interval between two LEDs adjacent to each other;

FIG. 6 is a partially exploded perspective view illustrating the roller and rack structure of the moving UV bar 20;

FIG. 7 is a partial sectional view for describing the structure of the sterilizer of the present invention;

FIG. 8 is a perspective view illustrating a left driving means of the sterilizer of the present invention;

FIG. 9 is a perspective view illustrating a right driving means of the sterilizer of the present invention; and

FIGS. 10, 11, and 12 are views illustrating the operation relation of a rack and pinions in the present invention.

### DETAILED DESCRIPTION OF THE INVENTION

Hereinafter, the present invention will be described in detail with reference to the accompanying drawings.

The present invention may be variously changed and have various forms, and an embodiment of the present invention will be described in detail hereinafter. However, this is not intended to limit the present invention to a specific disclosure form. Accordingly, it should be understood that the present invention includes all modifications, equivalents, or substitutions included in the spirit and scope of the present invention.

In addition, in each drawing, the size or thickness of each component is expressed to be exaggeratedly large (or thick) or small (or thin) or to have a simple form in consideration of the convenience of understanding. However, the protection scope of the present invention should not be interpreted to be limited thereto.

Terms used herein are only used to describe the specific embodiment (an aspect of the embodiment), and is not intended to limit the present invention. Singular expressions include plural expressions unless the context clearly indicates otherwise. It should be understood that terms such as “comprise” or “include” used herein specify the presence of features, steps, operations, components, or the combination thereof, but do not preclude the presence or addition of one or more other features, steps, operations, components, or the combination thereof.

Unless otherwise defined, all terms used herein, including technical or scientific terms, have the same meaning as commonly understood by those skilled in the art to which the present invention belongs. Terms defined in a commonly used dictionary should be interpreted as having meanings consistent with meanings in the context of related technologies, and should not be interpreted as ideal or excessively formal meanings unless explicitly defined in the present application.

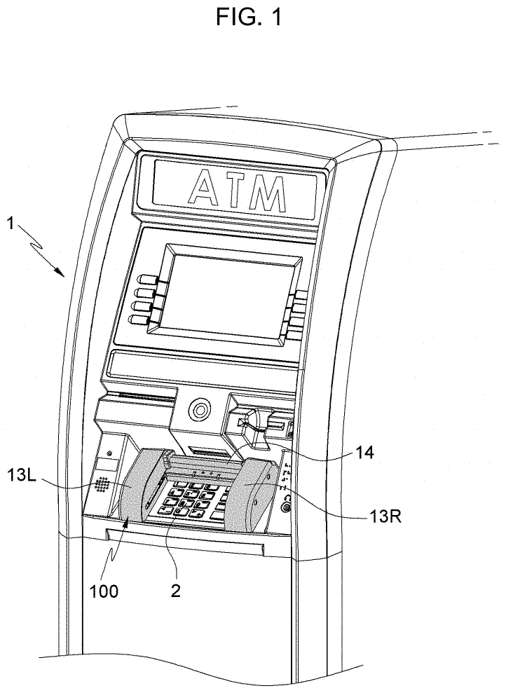

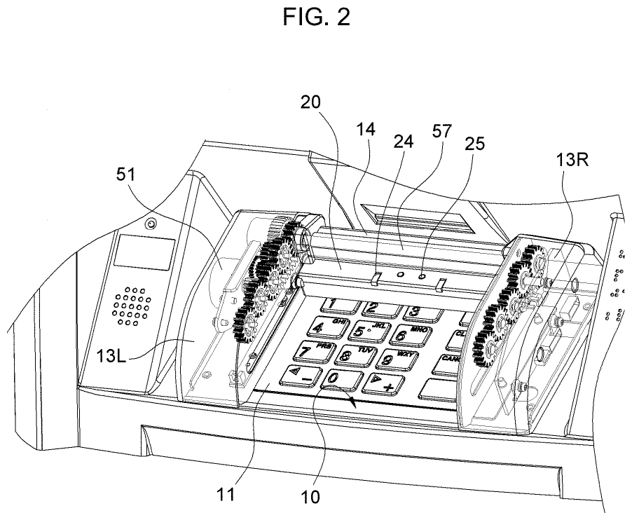

The present invention relates to a sterilizer attachable to an input pad of an automated device (an ATM, a kiosk, or a vending machine, etc.) currently in use. Particularly, as illustrated in FIGS. 1 and 2, the present invention relates to the sterilizer 100, wherein a surface of the input pad of an automated machine is sterilized while a moving UV bar 20 having a light source, in which multiple LEDs emitting UV-C are arranged, is vertically moved by the driving means.

A sterilizer having a fixed light source, which is not moved, has difficulty in achieving the purpose of blocking infection since sterilization is not performed in areas on which light is not directly emitted particularly due to the insufficient reflection of UV-C, and is not suitable for sterilizing, particularly, an EPP keypad having protruding buttons. However, in the sterilizer of the present invention, the light source 21 in which multiple high-power UV-C LEDs are integrated to a longitudinal bar is moved, so all areas including shadow areas that light cannot reach due to protruding buttons or protruding portions can be sterilized.

In the UV-C sterilizer 100 according to the present invention, an open portion 11 is provided in the center of a base plate 10 so that the input pad 2 of the automated machine 1 is exposed through the open portion 11, and the driving means is arranged inside the vertical covers 13L and 13R at opposite sides of the open portion 11 relative thereto and a horizontal cover 14 above the open portion 11.

The base plate 10 of the sterilizer according to the present invention is a component which is in close contact with or attached to the edges of the input pad of an automated machine which is being operated, and also functions to securely support the covers 13L, 13R, and 14 and left and right vertical frames 40L and 40R described hereinafter.

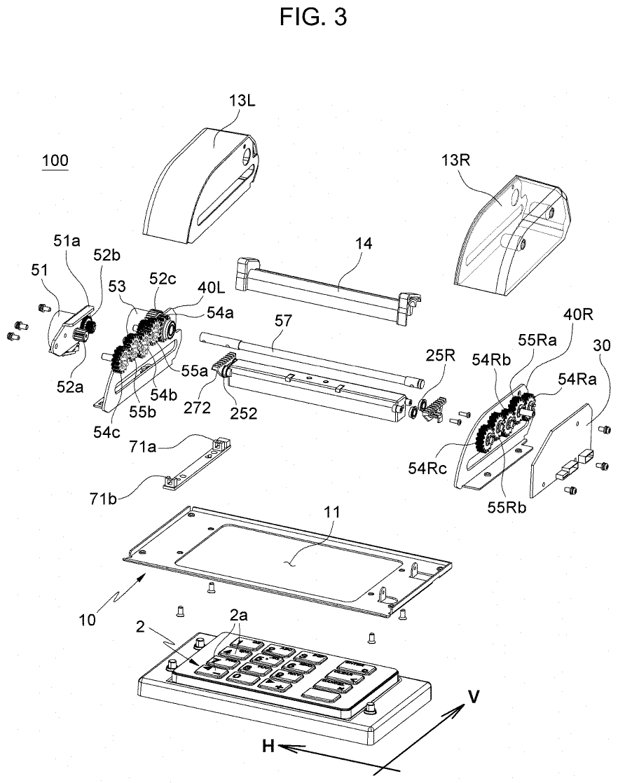

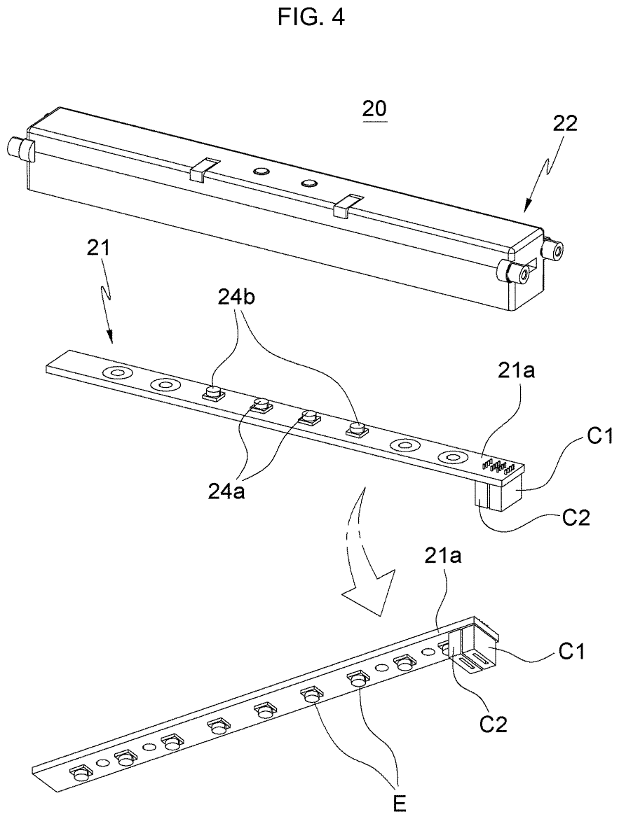

FIGS. 3 and 4 illustrate the embodiment of the present invention. Particularly, FIG. 3 is an exploded perspective view illustrating the structure of the sterilizer, and FIG. 4 is a perspective view illustrating the moving UV bar composed of the light source 21 in which each of the multiple high-power UV-C LEDs is arranged on the longitudinal bar and integrated thereto, and a protection cover 22. In FIG. 4, a reference numeral C1 refers to a cable socket for power supply, and a reference numeral C2 refers to a cable socket for control signal transmission.

In the UV-C LED according to the exemplary embodiment of the present invention, the UV-C LED emitting the light of a wavelength of 265 to 280 nm is applied in consideration of price, sterilization effect, and power consumption, etc. In order to solve the human-toxic problem of UV-C, safety measures described below are applied to the present invention.

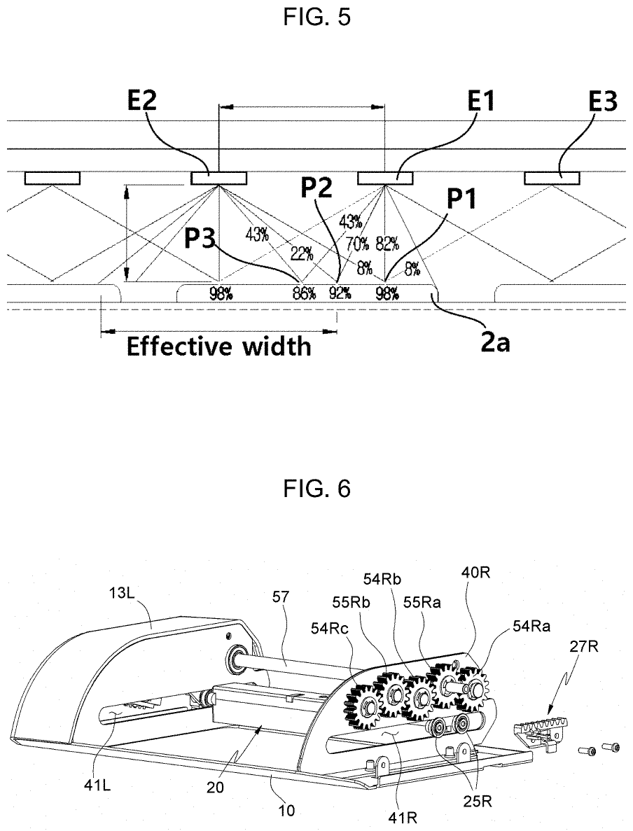

The radiant flux of light is inversely proportional to the square of a distance between the surface of a sterilization target and an LED light source, so sterilization efficiency is better as this distance is closer. However, as the distance is closer, the irradiation area of light decreases proportionally. Accordingly, as illustrated in FIG. 5, the distance between the surface of a sterilization target and the LED light source, the interval between the LEDs, and the average arrival rate of UV-C at a specific point on the surface of a sterilization target, etc. are required to be considered.

In the embodiment illustrated in FIG. 5, an interval between UV-C LEDs E1 and E2 adjacent to each other is set to be 12 mm, and a distance between the surface of a sterilization target 2a and the LED E1 is set to be 7 mm.

As illustrated in FIG. 5, the arrival rates of UV-C at points P1, P2, and P3 are 98%, 92%, and 86%, respectively (an average arrival rate 92%), and an effective sterilization width in which sterilization effect can be achieved by one LED is calculated to be about 17 mm. If time required to obtain the sterilization efficiency of 99% by one LED is 30 seconds, total time required to sterilize an input pad having a total movement length of 68 mm is calculated in the following equation: 30 seconds×68 mm/17 mm=120 seconds.

If the number of LEDs is doubled and arranged in two rows, time required for sterilization can be shortened in half.

Accordingly, the distance between the surface of a sterilization target and the LED light source, the setting of the interval between the LEDs in consideration of the average arrival rate of UV-C at the surface of a sterilization target, and the required transaction time for sterilizing are comprehensively considered, thereby minimizing the transaction time for sterilizing and maximizing the sterilization efficiency so that a next user does not wait for the time required for sterilization after a previous user finishes using the automated machine.

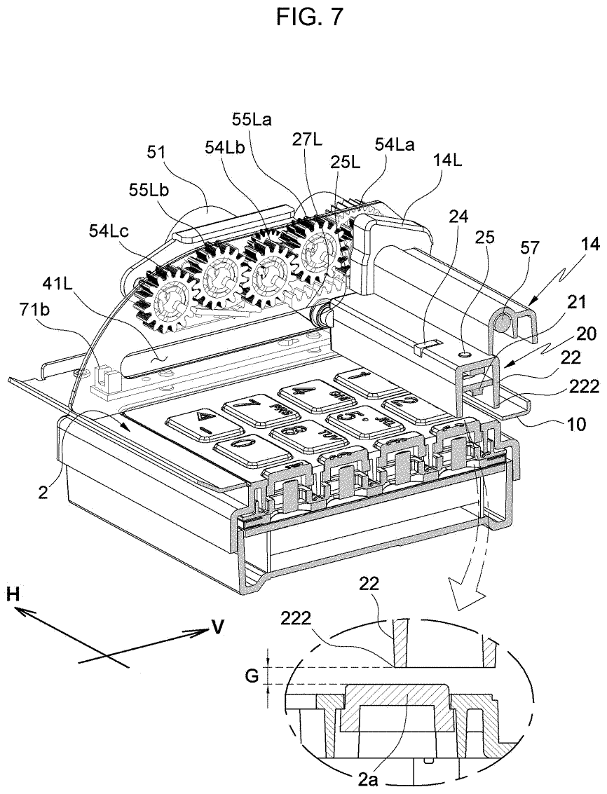

As illustrated in FIGS. 6 to 9, the driving means driving the moving UV bar 20 provided with UV-C the light source is configured to be compactly arranged inside a space defined by the covers 13L, 13R, and 14 and the base plate 10.

More particularly, the driving means of the present invention includes: a drive motor 51 provided at a left side of the open portion 11 of the base plate 10 relative thereto; left and right pinions 54L and 54R rotatably coupled to the left and right vertical frames 40L and 40R, respectively, and rotated by the drive motor 51 of the left side; and left and right racks 27L and 27R vertically moving the moving UV bar 20 by the rotation of the pinions 54L and 54R.

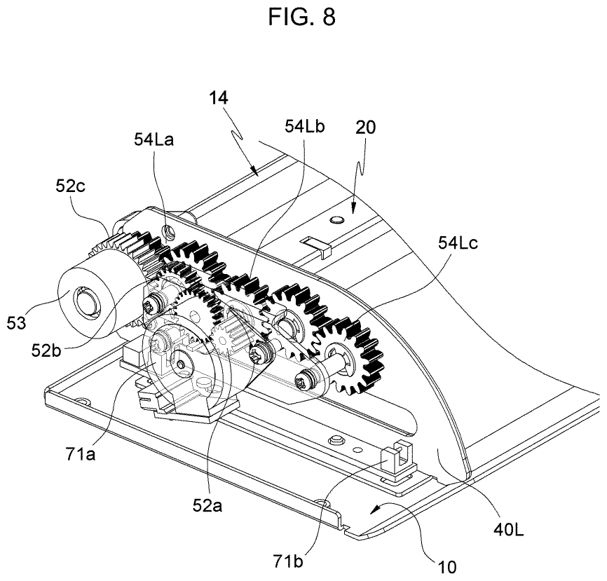

In FIGS. 3 and 7, a direction of a V arrow is referred to as an upside; a direction opposite thereto is referred to as a downside; a direction of an H arrow is referred to as a left side; and a direction opposite thereto is referred to as a right side. In FIG. 3, a reference numeral 51a refers to a bracket supporting the drive motor 51 and electric gears 52a, 52b, and 52c, and reference numerals 52a, 52b, and 52c refer to multiple electric gears converting the rotational speed of the drive motor 51 to an appropriate gear ratio and transmitting the appropriate gear ratio to the pinions 54L and 54R.

Rollers 25L and 25R are coupled to opposite ends of the moving UV bar 20, respectively. FIG. 6 is a partially exploded perspective view illustrating the roller and rack structure of the moving UV bar.

The left and right rollers 25L and 25R are fitted to slots 41L and 41R formed in the left and right vertical frames 40L and 40R, respectively, so that the moving UV bar 20 can be stably moved without shaking at the left and right ends thereof. The left and right vertical frames 40L and 40R having the slots 41L and 41R are vertically coupled to the opposite sides of the base plate 10, respectively, and support the shafts of the left and right pinions 54La, 54Lb, 54Lc, 54Ra, 54Rb, and 54Rc, and left and right idle gears 55La, 55Lb, 55Ra, and 55Rb.

The rollers 25L and 25R at opposite ends (left and right ends) of the moving UV bar 20 may include one or two rollers 25La and 25Lb, and 25Ra and 25Rb, respectively. The left and right racks 27L and 27R are coupled to the outer sides of the rollers 25L and 25R, respectively. To transmit a specific position (for example, a home position, a middle position, and maximally moved position, etc.) of the moving UV bar 20 to a controller, at least one sensor 71a or 71b is provided in the movement route of a left or right rack, so the position of the moving UV bar 20 can be checked.

The sterilizer according to the present invention is one of many ways to protect a user from UV-C light while sterilizing the input pad of the automated machine, and provides a solution for maximally securing the durability and mobile stability of the moving UV bar 20.

To make the driving means compact and to synchronize the rotational force, rotational speed, and phase of each of the left and right pinions 54L and 54R, one drive motor is preferably used, and a transmission shaft transmitting the driving force of the drive motor to a driving means located at a side opposite thereto is required. In FIGS. 3 and 6, a reference numeral 57 refers to the transmission shaft transmitting the rotational force and the rotational speed of the pinion 54La of a side driven by the drive motor to the pinion 54Ra of a side opposite thereto in synchronization with each other.

In the exemplary embodiment of the present invention, the transmission shaft 57 is installed in a horizontal direction at a side above the open portion 11, and is protected by the horizontal cover 14. When the transmission shaft 57 is arranged under the open portion 11, a user may be interrupted when manipulating the input pad.

As an important solution for protecting a user from UV-C light during the sterilization of the input pad 2 of the automated machine, the sterilizer according to the present invention includes the moving UV bar 20 composed of the light source 21 in which the multiple high-power UV-C LEDs are arranged at predetermined intervals on the longitudinal bar and integrated thereto, and the protection cover 22 preventing the light of the light source 21 from being emitted to directions other than a direction toward the surface of the input pad. Particularly, as illustrated in FIG. 7, a gap G defined between a lower edge 222 of the protection cover 22 and the surface of the input pad is configured to be smaller than the thickness of the fingers of a user.

Considering that the minimum finger thickness of an ordinary user manipulating the input pad of the automated machine is greater than 4 mm, in the present invention, a gap defined between the lower edge 222 of the protection cover 22 and the surface of the input pad is configured to be smaller than 4 mm to prevent the finger of a user from entering the gap, so the user can be protected from UV-C light. In the embodiment of the present invention, the gap defined between the lower edge 222 of the protection cover 22 and the surface of the input pad is 2 to 3 mm.

The sterilizer according to the present invention includes a torque limiter 53 as an important solution of protecting a user from UV-C light and preventing the sterilizer from being damaged due to an abnormal manipulation of a user during the sterilization of the input pad of the automated machine, the torque limiter being provided at an intermediate position of a power transmission system by which the rotational force is transmitted from the drive motor 51 to the pinions 54La and 54Ra.

When the movement of the moving UV bar 20 is restricted by the impact of a user while the moving UV bar 20 performs UV-C sterilization during the vertical movement, the moving UV bar 20 stops moving, and the drive motor 51 continues to rotate. In this case, the drive motor 51 or the power transmission system is overloaded, so a portion of a mechanical structure may be damaged.

When such a problem occurs, the torque limiter 53 functions to limit the rotational force of the drive motor 51 from being transmitted to the pinions any more. Accordingly, in the sterilizer of the present invention having the torque limiter 53, even when the movement of the moving UV bar 20 is restricted by a user while moving (during UV-C sterilization), a mechanical structure can be prevented from being damaged, and further, a signal can be transmitted to the controller such that the moving UV bar 20 is returned to a home position or is moved to a temporary position when an operational malfunction is determined to occur by a user so as to stop the operation of the moving UV bar 20.

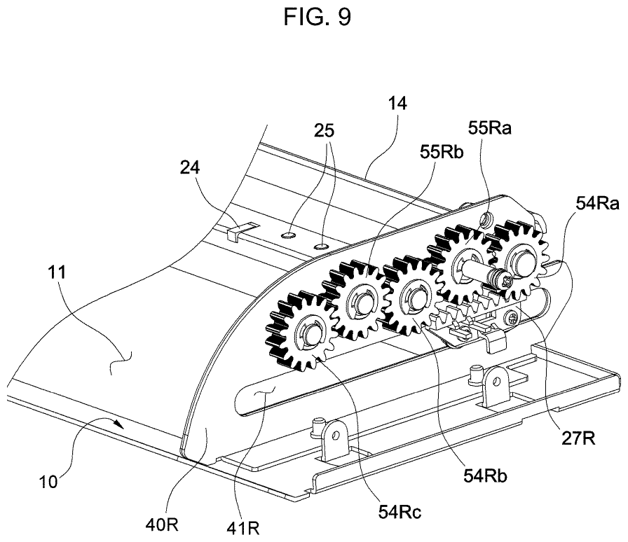

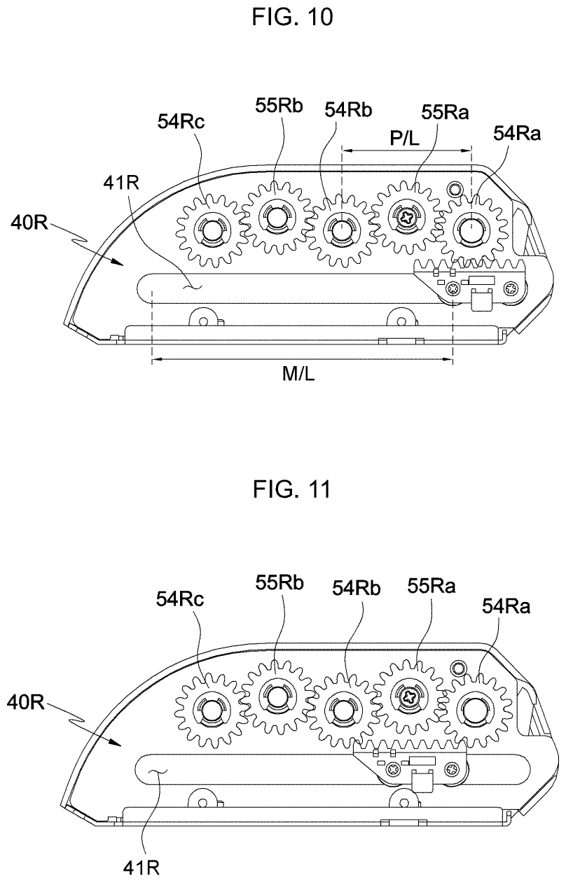

FIG. 8 is a perspective view illustrating a left driving means of the sterilizer of the present invention, and FIG. 9 is a perspective view illustrating a right driving means of the sterilizer of the present invention.

AS illustrated in FIGS. 8 and 9, when the drive motor 51 operated by the signal of the controller is rotated, the first electric gear 52a connected to the shaft of the drive motor is rotated, and the second electric gear 52b and the third electric gear 52c engaged with the first electric gear are sequentially rotated. The third electric gear 52c is connected to the torque limiter 53, and the torque limiter 53 is connected to a left first pinion 54La and the transmission shaft 57. The left first pinion 54La is connected to the right first pinion 54Ra by the transmission shaft 57, so the left first pinion 54La and the right first pinion 54Ra move the left and right racks 27L and 27R engaged with each tooth thereof while rotating at the same rotational force and rotational speed.

The racks 27L and 27R are coupled to the opposite ends of the moving UV bar 20; the left and right pinions are connected to each other by the transmission shaft 57 to rotate at the same rotational speed; and the rollers 25L and 25R are provided at opposite sides of the moving UV bar 20 to be guided along the slots 41L and 41R. Due to such a structure, the moving UV bar 20 according to the present invention can be vertically moved stably and smoothly even by one drive motor 51.

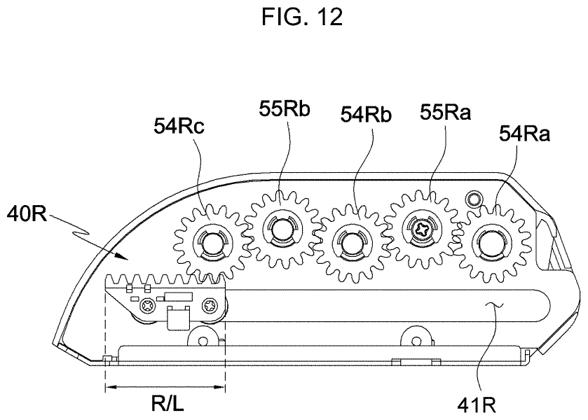

FIG. 10 is a view illustrating the operation relation of the rack and pinions in the present invention. As illustrated in FIG. 10, in the embodiment of the present invention, each of the left pinion and the right pinion is configured to include three pinions. The coupled structure of the pinions, idle gears, and rack of a left side is the same as the coupled structure of those of a right side, so the pinions and idle gears of the right side will be described below.

The right pinion is composed of the first pinion 54Ra, the second pinion 54Rb, and the third pinion 54Rc, which have the same heights and are provided by being spaced apart from each other at predetermined intervals.

A first idle gear 55Ra is provided to transmit a rotational force between the first pinion 54Ra and the second pinion 54Rb, and a second idle gear 55Rb is provided to transmit a rotational force between the second pinion 54Rb and the third pinion 54Rc while making the second pinion 54Rb and the third pinion 54Rc rotate in the same directions.

The first pinion 54Ra, the second pinion 54Rb, and the third pinion 54Rc are all rotated in the same directions by the first idle gear and the second idle gear.

FIG. 10 illustrates the position state of the rack and pinions when the moving UV bar 20 is located at a home position; FIG. 11 illustrates the position state of the rack and pinions when the moving UV bar 20 is moving; and FIG. 12 illustrates the position state of the rack and pinions at a position to which the moving UV bar 20 is maximally moved, that is, when the moving UV bar 20 arrives at a turning position.

As illustrated in FIGS. 10 to 12, the left pinion or the right pinion, or the left and right pinions according to the present invention are a plurality of pinions engaged with each other by idle gears, and move respective racks by being rotated in the same directions by the drive motor, and the length of each of the racks 27R is configured to be longer than a length P/L between shafts of two pinions adjacent to each other and to be shorter than a rectilinear movement stroke M/L of the rack.

Due to such a configuration, as illustrated in FIG. 11, the rack 27R being moved by being engaged with the pinion 54Ra continues to rectilinearly move by being engaged with the pinion 54Rb adjacent thereto before the engagement of the rack 27R with the pinion 54Ra ends.

Accordingly, since even with the short length R/L of the rack, a sufficiently long movement stroke M/L of the rack can be obtained, the length of the rack can be reduced to make the driving means compact. Here, the distance P/L between the shafts of two pinions adjacent to each other is naturally shorter than the length R/L of the rack.

To protect a user, the sterilizer according to the embodiment of the present invention may further include a controller receiving information on whether or not a user is in front of the automated machine from the automated machine, and controlling such that the sterilizer is operated when there is no user.

The controller may be configured to control the sterilizer such that an electric current is applied to the UV-C LEDs only when each part of a driving mechanism, a control board 30, and a software is recognized to be in a normal state. Accordingly, the controller is configured to apply an electric current to the UV-C LEDs only when each part of the sterilizer of the present invention is in a normal state, and to cut the application of an electric current to the UV-C LEDs when any one part of sterilizer is not in the normal state so that the safety of a user can be improved.

The controller may be configured to control the sterilizer such that a status result value of the sterilizer is transmitted to the automated machine after checking whether each part thereof is in a normal state such that information on whether the sterilizer is operated or the maintenance of the sterilizer can be maintained inside the operating system of the entirety of the automated machine.

In the sterilizer according to the exemplary embodiment of the present invention, the operation state of the sterilizer is preferably displayed for a user on the upper surface of the protection cover 22 of the moving UV bar 20 by two color LEDs 24a to inform the user that a sterilization operation is in progress so that the user can wait until the start of use of the automated machine.

In addition, the sterilizer according to the exemplary embodiment of the present invention is provided with a purple-colored LED 24b in parallel with the UV-C LEDs, and allows a user who cannot see the light of a UV-C wavelength to recognize the emission of UV-C light through the light of the above-described purple-colored LED 24b so that the user is careful.

The sterilizer according to the present invention described so far is specifically described to have various solutions applied thereto to prevent UV-C light from being harmful to users.

So far, the sterilizer according to the present invention has been described by focusing on the exemplary embodiment, but the sterilizer according to the present invention may be modified in various forms.

What is claimed is:

1. A sterilizer attachable to an input pad of an automated machine, the sterilizer sterilizing a surface of the input pad of an automated machine while vertically moving a moving UV bar having a UV light source by using a driving means, the sterilizer comprising:
a moving UV bar having a light source in which multiple high-power UV-C LEDs are arranged at predetermined intervals on a longitudinal bar and integrated thereto, and a protection cover preventing light of the light source from being emitted in directions other than a direction toward a surface of an input pad;
a base plate having an open portion corresponding to the input pad of an automated machine and attachable to edges of the input pad;
a horizontal cover provided in a horizontal direction above the open portion and a vertical cover provided at each of opposite sides of the open portion to protect a driving means; and
the driving means configured to be compactly arranged inside a space defined by the covers and the base plate,
wherein a gap defined between a lower edge of the protection cover and the surface of the input pad is configured to have a size to prevent a finger of a user from entering the gap.

2. The sterilizer of claim 1, wherein the driving means comprises:
left and right rollers rotatably coupled to opposite ends of the moving UV bar;
left and right vertical frames having a slot provided in each of the vertical frames, the slots of the frames guiding the rollers, and vertically coupled to opposite sides of the base plate, respectively;
left and right pinions rotatably coupled to the left and right vertical frames, respectively, and rotated by a drive motor provided at a side;
a transmission shaft synchronizing rotational speeds and phases of the left and right pinions; and
left and right racks coupled to outer sides of the left and right rollers, respectively, and vertically moving the moving UV bar by rotation of the pinions.

3. The sterilizer of claim 2, wherein the left pinion or the right pinion, or the left and right pinions are a plurality of pinions engaged with each other by idle gears, and move respective racks by being rotated in same directions by the drive motor, and
a length of each of the racks is configured to be longer than a length between shafts of two pinions adjacent to each other and to be shorter than a rectilinear movement stroke of the rack, so that the rack being moved by being engaged with a pinion continues to rectilinearly move by being engaged with a pinion adjacent thereto before the engagement of the rack with the earlier pinion ends.

4. The sterilizer of claim 2, further comprising:
a torque limiter provided at an intermediate position of a power transmission system by which a rotational force is transmitted from the drive motor to the pinions, the torque limiter preventing damage of components of the automated machine when the movement of the moving UV bar is restricted by impact of a user while the moving UV bar performs UV-C sterilization during vertical movement thereof, and controlling so that the moving UV bar is returned to an original position thereof or is moved to a temporary position after the user determines that an operational malfunction occurs when the moving UV bar does not reach a sensor at a specific position by a predetermined time.

5. The sterilizer of claim 3, further comprising:
a controller controlling operations of the driving means and the UV-C LEDs,
wherein the controller receives information on whether or not a user is in front of the automated machine from the automated machine, and controls such that an electric current is applied to the driving means and the UV-C LEDs when there is no user.
